(19) United States
(12) Patent Application Publication
(10) Pub. No.: US 2008/0269881 A1
(43) Pub. Date: Oct. 30, 2008

(54) Intraocular Lens with Asymmetric Haptics

(76) Inventors: Michael J. Simpson, Arlington, TX (US); James M. Scott, Millsap, TX (US); Son Trung Tran, Arlington, TX (US)

Correspondence Address: ALCON, IP LEGAL, TB4-8, 6201 SOUTH FREEWAY, FORT WORTH, TX, 76134, US

(21) Appl. No.: 11/741,854

(22) Filed: Apr. 30, 2007

(51) Int. Cl. A61F 2/16

(52) U.S. Cl. 623/6.17

(57) ABSTRACT

Asymmetric intraocular lenses (IOLs) are disclosed in which the centration of the optic and the pupil can be adjusted in order to reduce dsyphotopsia and/or the perception of dark shadows. For example, IOLs with uneven haptics are disclosed such that the center of the optic (i.e., the optical axis) is offset from a centerline of the overall device.

### BACKGROUND

The present invention relates generally to intraocular lenses (IOLs), and particularly to IOLs that provide a patient with an image of a field of view without the perception of visual artifacts in the peripheral visual field.

The optical power of the eye is determined by the optical power of the cornea and that of the natural crystalline lens, with the lens providing about a third of the eye's total optical power. The process of aging as well as certain diseases, such as diabetes, can cause clouding of the natural lens, a condition commonly known as cataract, which can adversely affect a patient's vision.

Intraocular lenses are routinely employed to replace such a clouded natural lens. Although such IOLs can substantially restore the quality of a patient's vision, some patients with implanted IOLs report aberrant optical phenomena, such as halos, glare or dark regions in their vision. These aberrations are often referred to as “dsyphotopsia.” In particular, some patients report the perception of dark shadows, particularly in their temporal peripheral visual fields. This phenomenon is generally referred to as “negative dsyphotopsia.”

Accordingly, there is a need for enhanced IOLs, especially IOLs that can reduce dysphotopsia, in general, and the perception of dark shadows or negative dysphotopsia, in particular.

### SUMMARY OF THE INVENTION

The present invention generally provides asymmetric intraocular lenses (IOLs) that alleviate, and preferably eliminate, dysphotopsia and/or the perception of dark shadows that is reported by some patients, in whose eyes conventional IOLs are implanted. In one embodiment, IOLs with uneven haptics are disclosed such that the center of the optic (i.e., the optical axis) is offset from the centerline of the overall device in one or both dimensions orthogonal to the optical axis.

The present invention is based, in part, on the discovery that the shadows perceived by IOL patients can be caused by a double imaging effect when light enters the eye at very large visual angles. More specifically, it has been discovered that in many conventional IOLs, most of the light entering the eye is focused by the cornea and the IOL onto the retina, but some of the peripheral light misses the IOL and it is focused only by the cornea. This leads to the formation of a second peripheral image. Although this image can be valuable since it extends the peripheral visual field, in some users it can result in the perception of a shadow-like phenomenon that can be distracting for some lens users.

To reduce the potential complications of cataract surgery, designers of modern IOLs have sought to make the optical component (the “optic”) smaller (and preferably foldable) so that it can be inserted into the capsular bag with greater ease following the removal of the patient's nature crystalline lens. The reduced lens diameter, and foldable lens materials, are important factors in the success of modern IOL surgery, since they reduce the size of the corneal incision that is required. This in turn results in a reduction in corneal aberrations from the surgical incision, and since often no suturing is required. The use of self-sealing incisions results in rapid rehabilitation and further reductions in induced aberrations. However, a consequence of the optic diameter choice is that the IOL optic may not always be large enough (or may be too far displaced from the iris) to capture all of the light entering the eye.

Moreover, the use of enhanced polymeric materials and other advances in IOL technology have led to a substantial reduction in capsular opacification, which has historically occurred after the implantation of an IOL in the eye, e.g., due to cell growth. Surgical techniques have also improved along with the lens designs, and biological material that previously could affect light near the edge of an IOL, and in the region surrounding the IOL, no longer does so. These improvements have resulted in a better peripheral vision, as well as a better foveal vision, for the IOL users. Though a peripheral image is not seen as sharply as a central (axial) image, peripheral vision can be very valuable. For example, peripheral vision can alert IOL users to the presence of an object in their field of view, in response to which they can turn to obtain a sharper image of the object. In some IOL users, however, the enhanced peripheral vision can lead to, or exacerbate, the perception of peripheral visual artifacts, e.g., in the form of shadows.

Dysphotopsia (or negative dysphotopsia) is often observed by patients in only a portion of their field of vision because the nose, cheek and brow block most high angle peripheral light rays—except those entering the eye from the temporal direction. Moreover, because the IOL is typically designed to be affixed by haptics to the interior of the capsular bag, errors in fixation or any asymmetry in the bag itself can exacerbate the problem—especially if the misalignment causes more peripheral temporal light to bypass the IOL optic.

In one aspect of the invention, asymmetric haptics are disclosed to permit centering of an IOL optic on the eye's optical axis (e.g., on the center of the iris) even if the capsular bag is irregular in shape or location. In another aspect, the asymmetric designs can be used to provide a degree of decentration such that the IOL is displaced slightly in the nasal direction to assist in the capture of temporal peripheral light rays. Decentration of the IOL's optic axis in the nasal direction vis-à-vis the eye's pupil can be from about 0.1 to about 2 mm, preferably from about 0.1 to about 1.5 mm and most preferable from about 0.5 to about 1 mm.

In another aspect of the invention, methods of treatment are disclosed, whereby an eye surgeon can assess the need for iris alignment (or decentration) and then select an IOL having a desired degree of longitudinal and/or latitudinal asymmetry for implantation. The IOL is preferably folded and inserted into the eye in the folded state. Following passage through the sclera and into the capsular bag, the IOL is unfolded and rotated to the desired orientation to ensure alignment with the center of the iris. Alternatively, the surgeon can choose an orientation that permits a degree of decentration, e.g., in the nasal direction.

Accordingly, a method of reducing visual artifacts in an eye with an implanted intraocular lens (IOL) is disclosed, in which an IOL is provided having an optic, a first haptic having first size dimensions, and a second haptic having second, different size dimensions, and the IOL is implanted into an eye of a patient. The method can further include the step of orienting the implanted IOL to adjust the alignment of the optic relative to the pupil and to the optical axis of the eye. In one embodiment, the method can be practiced by aligning the optic with the eye's pupil, e.g., by positioning the implanted IOL such that the optic is centered relative to the eye's pupil. Alternatively, the implanted IOL can be positioned such that the optic is decentered relative to the eye's pupil, e.g., offset in a nasal direction to reduce symptoms of negative dysphotopsia. Alternatively, the optic can be positioned in order to control both negative dysphotopsia and optical aberrations. Positioning of the IOLs of the present invention can be achieved by rotating the implanted IOL to achieve a desired location.

In yet another aspect of the invention, methods of manufacture are disclosed whereby two haptics of even size or shape are joined to opposite sides of an optic to form an asymmetric IOL. The method of manufacturing can include the steps of forming a first haptic having a first geometry, forming a second haptic with a second geometry that differs from the first geometry in at least one dimension, and joining the first and second haptics to an optic such that the assembly is adapted for use as an intraocular lens. The step of forming and joining can be done sequentially or they can be simultaneous, especially when the haptics and optic are made from the same material.

In certain embodiments, the asymmetric IOLs of the invention can also include one or more peripheral extensions that capture the light rays entering the eye at very large visual angles, and inhibit those rays from forming a secondary image. For example, in some embodiments, an IOL of the invention can include a peripheral extension that captures light rays entering the eye at large visual angles on the temporal side. (That is to say that upon implantation, the peripheral extension will extend in the nasal direction). Thus, in addition to centering the optic itself on the iris to minimize negative dysphotopsia, the peripheral extension can capture extreme peripheral rays that might otherwise still bypass the IOL (and/or rays that are misdirected due to internal reflections).

In some embodiments, the peripheral extension can scatter the incident light rays (e.g., via one or more textured surfaces) so as to reduce the visibility of the shadow. In other embodiments, the peripheral portion can include one or more opaque surfaces that substantially inhibit the incident peripheral light rays from reaching the retina. In yet other embodiments, the IOL's peripheral portion can focus the incident peripheral rays towards the shadowlike phenomenon to reduce its visibility.

While the asymmetric IOLs of the present invention may increase the overall length of the device, the width of the IOL can be maintained such that, upon folding, the IOL can be readily inserted into the eye through a conventional small incision. Even if the width is also increased (to provide an offset in this dimension as well), the additional width can be sufficiently small to permit folded insertion.

In some such embodiments, each of the anterior and posterior surfaces can be characterized by two orthogonal meridians one of which exhibits a radial extension or flange relative to the optical axis greater than about 3.5 mm and other exhibits a smaller radial extension (e.g., less than about 3.1 mm).

Further understanding of various aspects of the invention can be obtained by reference to the following detailed description in conjunction with the associated drawings, which are described briefly below.

### BRIEF DESCRIPTION OF THE DRAWINGS

FIG. 1 is a schematic anterior view of an intraocular lens (IOL) according to the invention showing an optic with a longitudinal offset,

FIG. 2 is a schematic anterior view of another intraocular lens (IOL) according to the invention showing an optic with a latitudinal offset,

FIG. 3 is a schematic anterior view of a further embodiment of an intraocular lens (IOL) according to the invention showing an optic with a longitudinal offset and peripheral extensions,

FIG. 4 is a schematic illustration of eye showing a conventional IOL placement in an irregular capsular bag and the consequent misalignment of the optic and iris.

FIG. 5 is a schematic illustration of eye showing placement of an IOL according to the invention to provide correct alignment of the optic and iris.

### DETAILED DESCRIPTION

The term “intraocular lens” and its abbreviation “IOL” are used herein interchangeably to describe lenses that are implanted into the interior of the eye to either replace the eye's natural lens or to otherwise augment vision regardless of whether or not the natural lens is removed. Phakic lenses, for example, are examples of lenses that may be implanted into the eye without removal of the natural lens. The term “longitudinal” is used herein to refer to the length dimension of the IOL, e.g., in the general direction of the haptics—typically the longer dimension of the IOL. Similarly, the term “latitudinal” is used herein to refer to the width dimension of the IOL, e.g., in a direction generally perpendicular to the haptics—typically the shorter dimension of the IOL.

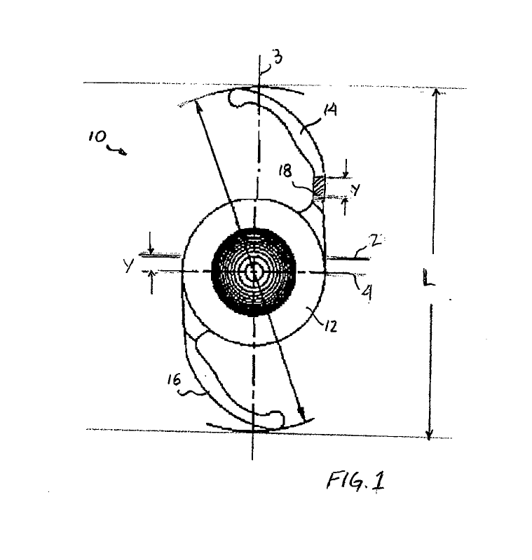

FIG. 1 shows an intraocular lens (IOL) 10 having an optic 12, a first haptic 14 and a second haptic 16. Unlike conventional IOLs in which the haptics are substantially identical, haptic 14 includes a longitudinal offset region 18 which serves to lengthen haptic 14 vis-à-vis haptic 16. In FIG. 1, the IOL 10 has an overall length L and a longitudinal center line 2. The offset 18 shifts the center of the optic 12 (and its corresponding center line 4) by a distance y such that the optic 12 is longitudinally decentered.

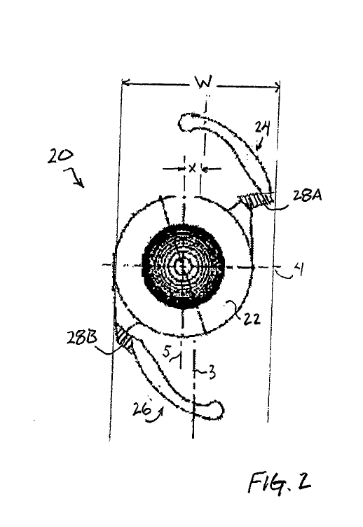

FIG. 2 shows another embodiment of an intraocular lens (IOL) 20 according to the invention again including an optic 22, as well as a first haptic 24 and a second haptic 26, having a latitudinal offset regions 28A and 28B, respectively. Offset region 28A served to extend haptic 24 outward, while offset region 28B serves to draw haptic 26 inwards. The net result is the overall width W of the IOL 20 defines a latitudinal center line 3 that is shifted vis-à-vis the optic center line 5. The offsets 28A and 28B shift the center of the optic 22 (and its corresponding center line 5) by a distance x such that the optic 22 is latitudinally decentered.

The optic and haptics described above can be made as separate pieces attached together or as one-piece of a polymeric material such as acrylates, e.g., polymethylmethacrylates (PMMAs), or polypropylenes or other foldable materials such as silicones, hydrogels or acrylics. It is usually desirable that the IOL be foldable for insertion to the eye through a small incision and then unfolded when positioned in the eye.

The optic is preferably formed of a biocompatible material, such as soft acrylic, silicone, hydrogel, or other biocompatible polymeric materials having a requisite index of refraction for a particular application. For example, in some embodiments, the optic can be formed of a cross-linked copolymer of 2-phenylethyl acrylate and 2-phenylethyl methacrylate, which is commonly known as Acrysof®.

Generally speaking, the haptics described above serve to secure the IOL within the capsular bag and prevent IOL migration. Stability is therefore an important factor to avoid the need for surgery to reposition the lens. Each haptic includes a base adjacent to the optic, a distal foot portion and an intermediate portion connected between the base and the distal foot. The haptics can also be formed of a suitable biocompatible material, such as polymethacrylate, polypropylene and the like. While in some embodiments, the haptics can be formed integrally with the optic, in other embodiments, the haptics are formed separately and then attached to the optic. It should be appreciated that various haptic designs for maintaining lens stability and centration are known in the art, including, for example, C-loops, J-loops, and plate-shaped haptic designs. The present invention is readily employed with any of these haptic designs.

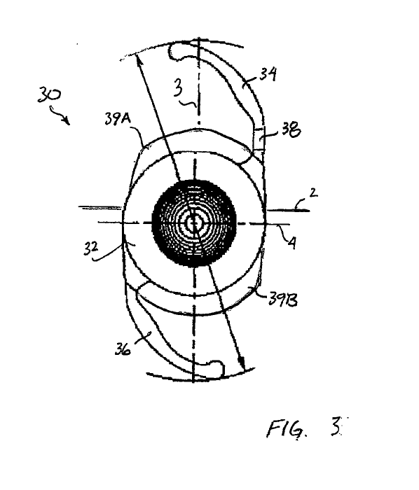

FIG. 3 is a schematic anterior view of a further embodiment of an intraocular lens (IOL) 30 according to the invention showing an optic 32 with peripheral extensions 39A and 39B, and haptics 34 and 36. Similar to the embodiment shown in FIG. 1, haptic 34 differs from haptic 36 by longitudinal extension 38, again causing the optic 32 to be longitudinally decentered. In this embodiment, either the peripheral optic extension 39A or peripheral optic extension 39B is adapted to be positioned, upon implantation of the IOL in the eye, on the nasal side of the eye such that the peripheral light rays entering the eye from the temple side would be incident thereon—the nose, eyebrows and cheeks typically block the entry of peripheral light rays from other directions into the eye.

Accordingly, the asymmetric haptics of the IOL 30 shown in FIG. 3 permit centration of the optic 32 relative to the iris even if the capsular bag geometry would otherwise cause a deviation. Alternately, the asymmetric haptics permit decentration of the optic 32 relative to the iris (e.g., in the nasal direction), if desired. In either case, the peripheral extensions 39A and/or 39B can capture and redirect or absorb the high angle peripheral light rays that may enter eye and still miss a conventional optic.

With reference to FIGS. 1-3, the IOLs 10, 20 and 30 can each be foldable, e.g., about an axis 3, to facilitate its implantation in the eye. Even with longitudinal asymmetry and/or peripheral extension the size of the IOL is only increased in length, not width, thus allowing a folded IOL to be readily inserted through a small incision into the eye. The latitudinal extension illustrated in FIG. 2 may increase the folded width of IOL 20 but this can be minimized by design of the haptics.

More particularly, during cataract surgery, a clouded natural lens can be removed and replaced with the IOL 10, 20 or 30. An incision is first made in the cornea to allow other instruments to enter the eye. The anterior lens capsule can be accessed via that incision to be cut in a circular fashion and removed from the eye. A probe can then be inserted through the corneal incision to break up the natural lens via ultrasound, and the lens fragments can be aspirated. An injector can be employed to place the IOL in a folded state in the original lens capsule. Upon insertion, the IOL can unfold and its haptics can anchor it within the capsular bag.

Once implanted in a patient's eye, the IOL can form an image of a field of view with its peripheral portion receiving peripheral light rays entering the eye at large visual angles and refracting those rays towards the image, thereby controlling visibility of a secondary image that could lead to perception of dark shadows (that is, the peripheral portion inhibits dysphotopsia). The term “large visual angles,” as used herein, refer to angles relative to the visual axis of the eye that are greater than about 50 degrees, and are typically in a range of about 50 degrees to about 80 degrees relative to eye's visual axis.

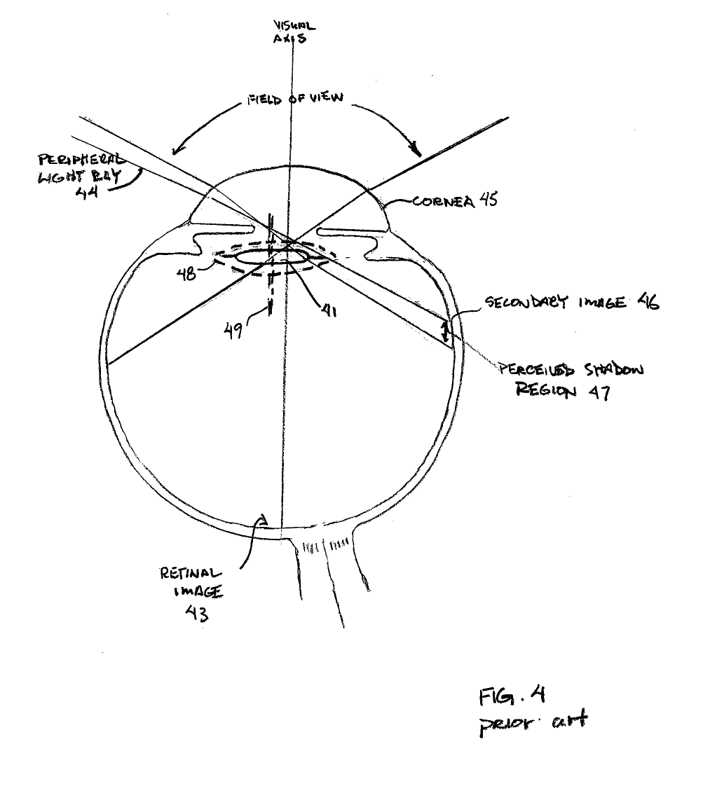

To further illustrate the role of asymmetric IOLs in inhibiting dysphotopsia, FIG. 4 shows a conventional IOL 41 (with identical haptics) implanted in an eye 42. Typically, the haptics serve to center the IOL within the capsular bag 48. If the bag geometry is irregular or if the haptics are otherwise misplaced during implantation, this can result in a misalignment of the IOL optical axis 49 relative to the optical axis OA of the eye. The conventional IOL 41 will form an image 43 of a field of view by focusing a plurality of light rays entering the eye onto the retina. Peripheral light rays (such as ray 44) that enter the eye at large visual angles are refracted by the cornea 45 but miss the IOL 41, at least in part, due to its misalignment. These high-angle, peripheral rays reach the retina at a location separated from the image 43 to form, in many cases, a secondary image 46. This can result in the perception of a shadow-like phenomenon 47 (negative dysphotopsia) between those images by the patient. Other factors that contribute to these phenomena include depth of IOL implantation (the distance between the IOL and the iris) and the patient's mean pupil size.

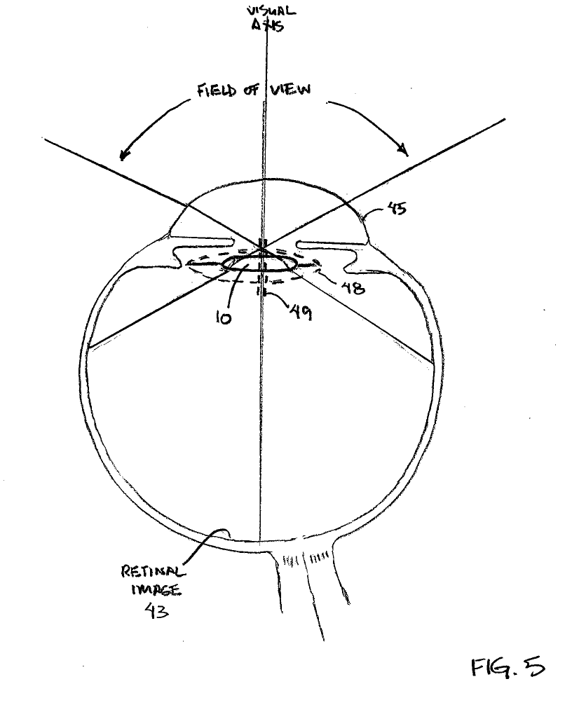

FIG. 5 shows an IOL 10 according to the invention implanted in the eye 42. (It should be clear that any of the IOLs described herein, including but not limited to those illustrated as IOL 20 and IOL 30 can likewise be used to address the problem.) Because of the asymmetric nature of the haptics in the IOLs of the present invention, capsular bag irregularities and other alignment problems can be alleviated by simple manipulation of the IOL during implantation. Rotation of the IOL within the capsular bag allows the surgeon to select an orientation that either ensures centration of the IOL optic relative to the iris of the eye 42 or, if desired, shifts the center of the IOL in the nasal direction (towards the patient's nose).

With further reference to FIG. 3, the peripheral extensions 39A and 39B of the IOL 30 can direct peripheral light rays entering eye at large visual angles towards the retina such that they form a single image together with the rays focused by the central portion. In other words, the peripheral extension prevents the peripheral light from forming a secondary image on the retina. Instead, the optic 32 and the peripheral extension(s) of the IOL can function as a single focusing unit to form a single image of a field of view by focusing the light rays entering the eye over a range of visual angles, including those entering the eye from the temporal side at large visual angles. In this manner, the IOL 30 can inhibit dysphotopsia.

In other embodiments, rather than utilizing a focusing peripheral portion, the IOL of the invention can include a peripheral extension characterized by at least one textured, opaque, and/or translucent surface that inhibits formation of a secondary image by light rays entering the eye at large visual angles. In this embodiment, the peripheral portion of the optic is textured in order to cause scattering of the peripheral light rays entering the eye at large visual angles so as to ensure that those rays would not form a discernible image on the retina whose separation from a primary image formed by the optic's central portion would lead to the perception of dark shadows. More specifically, one or both surfaces of the peripheral extension(s) can exhibit surface undulations (that is, the peripheral portion of the surface is textured) with amplitudes typically of the order of wavelengths of the visible light, e.g., less than about 1 micron. These surface undulations can cause scattering of peripheral light rays incident thereon, and hence inhibit formation of an image by those rays. Alternatively, the scattered rays can be redirected preferentially to reduce the perception of dark shadows by illuminating darker regions of the overall retinal image.

Alternately, the peripheral extensions can be opaque to visible radiation so as to significantly reduce, and preferably eliminate, the fluence of peripheral light that passes through it to reach the retina. More preferably, the opaque peripheral portion prevents such peripheral rays, e.g., via absorption, from reaching the retina. The term “opaque” as used herein, refers to an opacity that would result in a reduction in the intensity of the visible radiation, e.g., radiation with wavelengths in a range about 380 nm to about 780 nm, by more than about 25%, and preferably by more than about 50%, and most preferably by 100%. By way of example, in many embodiments, the intensity of the incident light passing through the opaque peripheral portion is reduced by a factor greater than about 25 percent and more preferably greater than about 50 percent.

In some embodiments, the peripheral extensions can include a Fresnel lens for directing light to the retinal reduced intensity (dark shadow) region. By way of example, a Fresnel lens can be disposed on an anterior surface of the peripheral extension and is adapted to direct light rays incident thereon to the retinal dark (shadow) region. To this end, in many embodiments, the Fresnel lens can have an optical power less than the optical power of the cornea alone and/or the optical power of the cornea and the IOL's optic. For example, the optical power of the Fresnel lens can be about one-half of the optical power of the cornea alone and/or that of the cornea and the IOL's optic.

In some embodiments, at least one of the anterior or posterior surfaces of the IOLs of the present invention can also exhibit an asphericity designed to ameliorate, and preferably prevent, spherical aberration effects that may arise from focusing of the peripheral light rays by the peripheral extension(s). By way of example, the IOL can have at least one aspheric surface characterized, e.g., by a conic constant in a range of about −10 to about −100, or in a range of about −15 to about −25. Further, in some cases, at least one surface of the IOL 94 can have a toric profile (i.e., a profile characterized by two different optical powers along two orthogonal surface directions). Additional teachings regarding the use of aspheric and/or toric surfaces in IOLs, such as various embodiments discussed herein, can be found in U.S. patent application Ser. No. 11/000,728 entitled “Contrast-Enhancing Aspheric Intraocular Lens,” filed on Dec. 1, 2004 and published as Publication No. 2006/0116763, which is herein incorporated by reference in its entirety.

Again, with reference to FIGS. 1-3, the central portion (or central optic) of the IOLs of the present invention can provide a single optical power or it can include a diffractive structure so as to provide multi-focal vision, e.g., both a far-focus optical power and a near-focus power. For example, the base curvature of the optic 12, 22 or 32 (and any peripheral extensions) can be selected such that the IOL provides a desired far-focus optical power, e.g., in a range of about −15 D to about 34 D. A diffractive structure disposed on the anterior surface provides a near focus optical power, e.g., in a range of about 1 D to about 4 D. In one embodiment, the diffractive structure can include a plurality of diffractive zones (as known in the art) that are separated from one another by a plurality of steps. The steps can be uniform or they can exhibit a decreasing height as a function of increasing distance from the optical axis. In other words, the step heights at the boundaries of the diffractive zones are “apodized” so as to modify the fraction of optical energy diffracted into the near and far foci as a function of aperture size (e.g., as the aperture size increases, more of the light energy is diffracted into the far focus). By way of example, the step height at each zone boundary can be defined in accordance with the following relation:

$$Step height = \frac{λ}{a ( {n}_{2} - {n}_{1} )} {f}_{apodize} \qquad Equation ( 1 )$$

wherein

λ denotes a design wavelength (e.g., 550 nm),

a denotes a parameter that can be adjusted to control diffraction efficiency associated with various orders, e.g., a can be selected to be 2.5;

n₂ denotes the index of refraction of the optic,

n₁ denotes the refractive index of a medium in which the lens is placed, and

ƒ_{apodize} represents a scaling function whose value decreases as a function of increasing radial distance from the intersection of the optical axis with the anterior surface of the lens. By way of example, the scaling function ƒ_{apodize} can be defined by the following relation:

$${f}_{apodize} = 1 - {( \frac{{r}_{i}}{{f}_{out}} )}^{3} . \qquad Equation ( 2 )$$

wherein

rᵢ denotes the radial distance of the i^{th} zone,

rₒᵤₜ denotes the outer radius of the last bifocal diffractive zone.

Other apodization scaling functions can also be employed, such as those disclosed in a co-pending patent application entitled “Apodized Aspheric Diffractive Lenses,” filed Dec. 1, 2004 and having a U.S. Ser. No. 11/000,770 (Pub. No. 2006/0116764), which is herein incorporated by reference.

In this exemplary embodiment, the diffractive zones are in the form of annular regions, where the radial location of a zone boundary (rᵢ) is defined in accordance with the following relation:

rᵢ²=(2i+1)λƒ Equation (3)

wherein

i denotes the zone number (i=0 denotes the central zone),

rᵢ denotes the radial location of the ith zone,

λ denotes the design wavelength, and

ƒ denotes an add power.

Those having ordinary skill in the art will appreciate that various changes can be made to the above embodiments without departing from the scope of the invention.

1. An intraocular lens (IOL), comprising
an optic,
a first haptic having first size dimensions, and
a second haptic having second size dimensions, wherein the first and second haptic dimensions differ.

2. The IOL of claim 1, wherein the first haptic has at least a length dimension that is greater than the length dimension of the second haptic.

3. The IOL of claim 2, wherein the optic is displaced from the center of the length dimension of the IOL by about 0.1 to about 5 mm.

4. The IOL of claim 2, wherein the optic is displaced from the center of the length dimension of the IOL by about 0.5 to about 2 mm.

5. The IOL of claim 2, wherein the decentered optic is displaced in the nasal direction upon implantation.

6. The IOL of claim 1, wherein the first haptic has at least a width dimension that is greater than the width dimension of the second haptic.

7. The IOL of claim 6, wherein the optic is displaced from the center of the width dimension of the IOL by about 0.1 to about 5 mm.

8. The IOL of claim 6, wherein the optic is displaced from the center of the width dimension of the IOL by about 0.5 to about 2 mm

9. The IOL of claim 6, wherein the decentered optic is displaced in the nasal direction upon implantation.

10. The IOL of claim 1, wherein said optic is foldable so as to allow its insertion into the eye.

11. The IOL of claim 1, which further comprises at least one peripheral extension capable of capturing high angle peripheral light rays that enter an eye upon implantation of the IOL into the eye.

12. The IOL of claim 11 wherein the at least one peripheral extension provides a maximum radial extension of the optic relative to an optical axis in a range of about 3.5 mm to about 4.5 mm.

13. The IOL of claim 11, wherein the peripheral extension comprises at least one textured surface adapted to cause scattering of the peripheral light rays.

14. The IOL of claim 11, wherein the peripheral extension comprises at least one surface that is opaque to visible radiation.

15. The IOL of claim 1, wherein said peripheral portion comprises at least one refractive surface adapted to redirect said peripheral light rays.

16. The IOL of claim 1, wherein the optic exhibits an asphericity characterized by a conic constant in a range of about −10 to about −100.

17. The IOL of claim 1, wherein the optic comprises a diffractive structure to provide at least a far-focus optical power and a near-focus optical power.

18. The IOL of claim 1, wherein the optic comprises a transparent polymeric material.

19. The IOL of claim 1, wherein the optic comprises at least one polymeric material selected from the group of acrylics, acrylates, methacrylates, silicones, polypropylenes and hydrogels.

20. The IOL of claim 1, wherein the optic comprises a copolymer of acrylate and methacrylate materials.

21. The IOL of claim 1, wherein the optic comprises a cross-linked copolymer of 2-phenylethyl acrylate and 2-phenylethyl methacrylate

22. The IOL of claim 1, wherein the haptic comprises a polymeric material.

23. The IOL of claim 1, wherein the optic and the haptic comprise a common polymeric material.

24. The IOL of claim 1, wherein the haptic comprises at least one polymeric material selected from the group of acrylics, acrylates, methacrylates, silicones, polypropylenes and hydrogels.

25. A method of manufacturing an asymmetric intraocular lens (IOL), the method comprising:
forming a first haptic having a first geometry,
forming a second haptic with a second geometry that differs from the first geometry in at least one dimension, and
joining the first and second haptics to an optic such that the assembly is adapted for use as an intraocular lens.

26. The method of claim 25, wherein the steps of forming the first and second haptics further comprises forming a first haptic that has at least a length dimension that is greater than the length dimension of the second haptic.

27. The method of claim 25, wherein the steps of forming the first and second haptics further comprises forming a first haptic that has at least a length dimension that is greater than the length dimension of the second haptic such that the optic is displaced from the center of the length dimension of the IOL by 0.5 to 10.0 micrometers.

28. The method of claim 25, wherein the steps of forming the first and second haptics further comprises forming a first haptic that has at least a length dimension that is greater than the length dimension of the second haptic such that the optic is displaced from the center of the length dimension of the IOL by 2.5 to 5.0 micrometers.

29. The method of claim 25, wherein the assembled IOL is configured such that the optic is displaced in the nasal direction upon implantation.

30. The method of claim 25, wherein the steps of forming the first and second haptics further comprises forming a first haptic that has at least a width dimension that is greater than the width dimension of the second haptic.

31. The method of claim 25, wherein the steps of forming the first and second haptics further comprises forming a first haptic that has at least a width dimension that is greater than the width dimension of the second haptic such that the optic is displaced from the center of the width dimension of the IOL by 0.25 to 5.0 micrometers.

32. The method of claim 25, wherein steps of forming the first and second haptics further comprises forming a first haptic that has at least a width dimension that is greater than the width dimension of the second haptic such that the optic is displaced from the center of the width dimension of the IOL by 0.5 to 3.0 micrometers.

33. The method of claim 25, wherein the step of forming the first haptic further comprises forming the first haptic from at least one materials selected from the group of acrylics, acrylates, methacrylates, silicones, polypropylenes and hydrogels.

34. The method of claim 25, wherein the method further comprises selecting an optic is foldable to join to the first and second haptics.

35. The method of claim 25, wherein the method further comprises selecting an optic having at least one peripheral extension capable of capturing peripheral lights that enter an eye upon implantation of the IOL into the eye.

36. The method of claim 25, wherein the method further comprises selecting an optic that comprises a transparent polymeric material.

37. The method of claim 25, wherein the method further comprises selecting an optic that comprises at least one polymeric material selected from the group of acrylics, acrylates, methacrylates, silicones, polypropylenes and hydrogels.

38. The method of claim 25, wherein the method further comprises selecting an optic that comprises a copolymer of acrylate and methacrylate materials.

39. The method of claim 25, wherein the method further comprises selecting an optic that comprises a cross-linked copolymer of 2-phenylethyl acrylate and 2-phenylethyl methacrylate

40. The method of claim 25, wherein the method further comprises selecting at least one haptic that comprises a polymeric material.

41. The method of claim 25, wherein the method further comprises selecting an optic and a haptic that comprise a common polymeric material.

42. The method of claim 25, wherein the method further comprises selecting a haptic that comprises at least one polymeric material selected from the group of acrylics, acrylates, methacrylates, silicones, polypropylenes and hydrogels.

44. A method of reducing visual artifacts in an eye with an implanted intraocular lens (IOL), the method comprising:
providing an IOL having an optic, a first haptic having first size dimensions, and a second haptic having second size dimensions,
implanting the IOL into an eye of a patient.

45. The method of claim 44 wherein the method further comprises positioning the implanted IOL such that the optic is aligned with an optical axis of the eye.

46. The method of claim 44 wherein the method further comprises positioning the implanted IOL such that the optic is aligned with the eye's pupil.

47. The method of claim 44 wherein the method further comprises positioning the implanted IOL such that the optic is centered relative to the eye's pupil.

48. The method of claim 44 wherein the method further comprises positioning the implanted IOL such that the optic is decentered relative to the eye's pupil.

49. The method of claim 48 wherein the method further comprises positioning the implanted IOL such that the center of the optic is offset in a nasal direction.

50. The method of claim 48 wherein the method further comprises positioning the implanted IOL such that the center of the optic is offset in a temporal direction.

51. The method of claim 44 wherein the method further comprises rotating the implanted IOL to achieve a desired position.
